(19) United States
(12) Patent Application Publication
(10) Pub. No.: US 2020/0289061 A1
(43) Pub. Date: Sep. 17, 2020

(54) INTRADURAL NEURAL ELECTRODES

(71) Applicants: Benjamin I. Rapoport, New York, NY (US); Athos Patsalides, New York, NY (US)

(72) Inventors: Benjamin I. Rapoport, New York, NY (US); Athos Patsalides, New York, NY (US)

(21) Appl. No.: 16/816,237

(22) Filed: Mar. 11, 2020

Related U.S. Application Data
(60) Provisional application No. 62/816,361, filed on Mar. 11, 2019.

(51) Int. Cl. A61B 5/00; A61B 5/04; A61N 1/05; A61L 31/02

(52) U.S. Cl. CPC A61B 5/6868; A61B 5/04001; A61N 1/0529; A61B 2090/376; A61B 5/6876; A61B 2562/0215; A61B 2560/063; A61L 31/022

(57) ABSTRACT

Described herein are systems and methods for deploying and recording electrophysiologic signals from electrode arrays located within the dura mater of the brain. The dura matter includes layers of connective tissue, or membrane, that surround the brain and spinal cord. The present disclosure relates to an endovascular electrode system deployed within the blood vessels located between layers of the dura mater, including, for example, the middle meningeal artery and its branches.

### REFERENCE TO CROSS-RELATED APPLICATIONS

The present disclosure claims priority to and the benefit of U.S. Provisional Application No. 62/816,361 entitled “Endovascular Electrophysiology (EEG) Systems, and Related Systems, Apparatus, and Methods” filed on Mar. 11, 2019, the contents of which are hereby incorporated by reference.

This application is also related to “ENDOVASCULAR ELECTROENCEPHALOGRAPHY (EEG) AND ELECTROCORTICOGRAPHY (ECoG) DEVICES, SYSTEMS AND METHODS” concurrently filed on Mar. 11, 2020, the contents of which are hereby incorporated by reference.

### TECHNICAL FIELD

The present disclosure is related to neural electrodes, and more particularly intradural neural electrodes.

### BACKGROUND

Several common disorders of the brain, spinal cord, and peripheral nervous system arise due to abnormal electrical activity in biological (neural) circuits. In general terms, these conditions may be classified into: (1) Conditions such as epilepsy, in which electrical activity is dysregulated, and recurrent activity persists in an uncontrolled fashion; (2) Conditions such as stroke or traumatic injury, in which an electrical pathway is disrupted, disconnecting a component of a functional neural circuit; and (3) Conditions such as Parkinson's disease, in which neurons in a discrete region cease to function, leading to functional impairment in the neural circuits to which the lost neurons belong.

When the electrical lesion is focal and relatively discrete the effective diagnosis and treatment of such conditions depends on precise localization of the lesion and, when possible, restoration of normal electrophysiologic function to the affected region.

Conventional techniques for localizing electrical lesions in the brain such as imaging techniques, electromagnetic recording techniques, electrocorticography (ECoG), depth electrodes, and deep brain stimulation techniques, each have specific limitations.

For example, imaging techniques such as magnetic resonance imaging (MRI) and computed tomography (CT) are non-invasive methods of examining brain tissue. These imaging techniques may be useful in detecting and localizing functional lesions, including strokes, anatomic abnormalities capable of causing seizures, and foci of neuronal degeneration. However, not all functional lesions can be detected using these imaging modalities because these techniques do not image electrical activity. Furthermore, these imaging techniques lack temporal resolution, and provide no mechanism for therapeutic electrophysiologic intervention.

Electromagnetic recording techniques such as electroencephalography (EEG) and magnetoencephalography (MEG) are also noninvasive techniques. EEG and MEG are able to provide temporal resolution of electrical activity in the brain, and thus often used for seizure detection. In conventional EEG, electrodes are positioned on the scalp. However, the spatial resolution of electromagnetic recording techniques is limited, both due to physical distance of electrodes from the brain, and by the dielectric properties of scalp and skull. Accordingly, the spatial resolution of EEG is better for superficial regions, and worse for neural activity deep within the brain. For example, seizures arising from anatomic abnormalities near the cortical surface are well localized by EEG and MEG.

Electrocorticography (ECoG), or intracranial EEG, is a form of electroencephalography that provides improved spatial resolution by placing recording electrodes directly on the cortical surface of the brain (in contrast to conventional EEG systems where electrodes are positioned on the scalp). ECoG is frequently used during neurosurgical procedures to map normal brain function and locate abnormal electrical activity. However, ECoG requires a craniotomy or a temporary surgical removal of a significant portion of the skull, in order to expose the brain surfaces of interest. This exposes patients to the attendant risks of brain surgery. Furthermore, while electrical activity near the cortical surface of the brain can be mapped with reasonable spatial resolution, electrical activity deep within the brain remains difficult to localize using ECoG.

“Depth electrodes” record electrical activity with high spatial and temporal precision. However, depth electrodes are configured to record only from small volumes of tissue (i.e., small populations of neurons). Further, the placement of depth electrodes requires the disruption of normal brain tissue along the trajectory of the electrode, resulting in irreversible damage or destruction of some neurons. As such, depth electrodes are conventionally placed surgically, in a hypothesis-driven manner (the technique is sometimes called “stereo-EEG” or “sEEG”), and the number of such electrodes that can be safely placed simultaneously is limited. Further, the electrode positions cannot be adjusted once the electrodes are placed, except for small adjustments to depth at the time of placement.

Deep brain stimulation (DBS) electrodes, a stimulating analog of recording depth electrodes, electrically stimulate brain regions with millimetric and/or sub-millimetric precision. They are implanted using minimally invasive surgical techniques, and can be effective in conditions such as Parkinson's disease and essential tremor, in which neuronal dysfunction is confined to small, discrete, and unambiguous regions of the brain. Some evidence suggests these techniques can be useful in treating epilepsy, as well as other disorders (not all of which are traditionally associated with focal brain lesions), including some psychiatric disorders and substance addiction. For example, symptoms of Parkinson's disease, arising from degeneration of dopamine-producing neurons in a well-defined region (the substantia nigra), can often be effectively modulated by precise stimulation of a millimetric nucleus (the subthalamic nucleus) using a small number of deep brain stimulation (DBS) electrodes.

Neural recording and stimulation techniques (including those discussed above) involve design trade-offs among a number of primary factors: (1) spatial resolution, (2) temporal resolution, (3) degree of invasiveness and collateral damage to normal brain tissue, and (4) optimization for electrical recording and/or electrical stimulation. An ideal electrophysiologic neural probe, should simultaneously provide optimal performance in all four of the above categories.

Diagnosis and treatment of functional electrophysiologic lesions in many brain regions remain challenging or intractable. In particular, deep brain regions are frequent sites of functional lesions, yet remain difficult to access systematically and minimally invasively. For example, the medial temporal lobe is a common site for seizure foci and the substantia nigra is the site of neuronal degeneration causing Parkinson's disease; both regions are several centimeters deep to the cortical surface. Accordingly, the conventional techniques discussed above such as imaging techniques, electromagnetic recording, ECoG, depth electrodes (sEEG), or deep brain stimulation are ill-equipped or imperfectly equipped to detect, localize, and treat these and related lesions in the brain.

### SUMMARY

The present disclosure is directed towards systems and methods for deploying and recording from electrode arrays positioned within the dura matter of the brain. In some embodiments, the electrode arrays are deployed with the blood vessels of the dura. The electrode arrays may be configured to record and/or stimulate neural tissue.

In some embodiments, an implantable medical device may include a linear array of electrodes configured for insertion between layers of a dura membrane of a brain, the linear array configured to record or stimulate electrical activity in brain tissue, and an insulated electrode trace connecting each of electrodes in the linear array. Optionally, each of the electrodes includes at least one of gold, silver, platinum, or platinum-iridium and each of the electrodes has a diameter between about 5 to 25 microns, or 25 to 250 microns. Additionally, the insulated electrode trace includes a diameter between about 6 to 35 thousandths of an inch in diameter and has a length between about 10 to 30 cm. Further, the diameter of the at least one of the insulated electrode trace or plurality of wire traces contained within the insulated electrode trace may taper proximate the electrode. In some embodiments, each electrode may be connected to a multiplexing element. In some embodiments, each electrode may be connected to an amplifier located outside of the body. In some embodiments, the linear array of electrodes may be positioned at the middle meningeal artery. In some embodiments, the linear array of electrodes may be positioned proximate the dural venous sinuses. In some embodiments, the surface of each of the electrodes is coated by poly(3,4-ethylenedioxythiophene) polystyrene sulfonate (PEDOT). In some embodiments the inter-electrode spacing distance between adjacent electrodes in the linear array is between about 5-500 microns. In some embodiments the insulated electrode trace connects to a long-term recording system or a stimulation system. In some embodiments, the insulated electrode trace includes a hydrophilic coating.

In some embodiments a method for operating intradural electrodes includes advancing a microwire through the external carotid artery to the middle meningeal artery, positioning electrodes between layers of the dura membrane via the middle meningeal artery, and recording or stimulating the brain tissue, where the microwire includes a linear array of electrodes configured for insertion between layers of the dura membrane of the brain, the linear array comprising a plurality of electrodes configured to record or stimulate electrical activity in brain tissue, and each of the electrodes may be connected to the linear array via an insulated electrode trace. In some embodiments, positioning electrodes within the layers of the dura further comprises applying fluroscopy or contrast dye techniques. In some embodiments, the method may include the step of moving the position of electrodes between layers of the dura membrane. In some embodiments the plurality of insulated electrode traces forms a bundle coated with hydrophilic coatings.

In some embodiments, a method for deploying intradural electrodes includes advancing a microwire through the external carotid artery to the middle meningeal artery, advancing an electrode array over the microwire to the middle meningeal artery, removing the microwire and recording or stimulating the brain tissue about the electrode array, where the electrode array includes a plurality of electrodes configured to record or stimulate electrical activity in brain tissue and the plurality of electrodes are on an insulated substrate. Optionally, positioning the electrode array within the middle meningeal artery may use least one of fluroscopy, and contrast dye techniques. Additionally, the method may include moving the position of the electrode array between layers of the dura membrane. In some embodiments, the electrode array may be implanted within the brain for weeks. In some embodiments, the method may include the step of advancing a microcatheter to recover and remove the electrode array.

### BRIEF DESCRIPTION OF THE DRAWINGS

FIG. 1 illustrates certain aspects of the anatomy of the head, including the relationship of the external carotid artery and its branches, some of which supply the dura, in accordance with embodiments of the present disclosure.

FIG. 2 illustrates a path for implantation of a neural electrode array in accordance with embodiments of the present disclosure.

FIG. 3 illustrates a neural electrode array positioned within the dura in accordance with embodiments of the present disclosure.

FIG. 4 illustrates a neural electrode array (mirowire) in accordance with embodiments of the present disclosure.

FIG. 5 illustrates a flowchart for a method for intradural recording and/or stimulation system in accordance with embodiments of the present disclosure.

### DETAILED DESCRIPTION

The present disclosure is directed towards systems and methods for deploying and recording electrophysiologic signals from electrode arrays located within the dura mater of the brain. The dura matter is a layer of connective tissue, or membrane, that surrounds the brain and spinal cord. The layer is typically tens to hundreds of microns in thickness, and in most regions comprises two sublayers that remain completely apposed to one another. The systems and methods described herein are distinct from existing systems and methods, in which recording electrodes are located on one side (outside) or the other (inside) of this tissue plane, but not actually within the dura mater itself (between its composite sublayers, for example). The present disclosure relates to a specific type of endovascular electrode system deployed within the blood vessels of the dura mater, referred to herein as “intra-dural.”

In some embodiments the disclosed systems and methods include electrode arrays, and more specifically microwires, designed for deployment within the blood vessels of the dura mater of the brain, including the middle meningeal artery and its branches.

The system further comprises intravascular leads and connectors routed through the vascular system, for transvascular and either subcutaneous connection to an implanted computer system, or external (transcutaneous) connection to an externally wearable computer system responsible for control of power, system control, data storage, and data transmission (wired or otherwise).

The system also comprises a mechanism for stable percutaneous cannulation of the superficial temporal artery, and deployment of intradural electrodes via branches of the external carotid artery, without entering the arteries (the internal carotid artery and its branches) that supply the brain itself.

Due to its anatomic size and positioning, the intradural space is not explored by conventional neurological imaging, recording and/or stimulation techniques. Embodiments built in accordance with the present disclosure access the intradural space via blood vessels through neuro-endovascular techniques.

The intradural space has not traditionally been exploited for the placement of implantable devices, as it is typically neither accessible through traditional surgical techniques nor an anatomic space of appreciable volume. The dura mater itself comprises two layers, an external layer (the “periosteal” layer, which lies apposed to the skull) and an internal layer (the “meningeal” layer, which lies closer to the surface of the brain). In most regions these two layers are completely apposed to one another, with no intervening space; the potential space between these two layers is known as the “epidural space.” Within the cranial cavity these two component layers separate essentially only to permit the passage of blood vessels (small to medium size arteries and veins, as well as the major venous structures of the brain, called the “venous sinuses”). Except where blood vessels run between the meningeal and periosteal layers, there is negligible volume in the cranial epidural space. For these reasons this space has not previously been considered a location amenable to placement of implantable devices.

As used herein, the term “intradural” refers to the space (or potential space) between the meningeal and periosteal layers, including and especially the vascular spaces between these two layers (such as the dural venous sinuses and the meningeal arteries). This is in contrast to conventional systems which may be positioned in the “epidural” and “epidural space,” the region external to the periosteal layer, between the periosteal layer and the bony inner surface of the skull.

The vessels in the intradural space, including the middle meningeal artery, are highly redundant, and can typically be occluded (if necessary) without clinical consequences, so access to these vessels is safe. Furthermore, these vessels can be accessed in a manner that relies only on the external carotid artery and its branches, bypassing the common and internal carotid arteries, which supply the brain, so as to minimize any potential risk of brain-related complications (such as embolic stroke or damage to an artery supplying the brain). While conventional neural electrodes are typically placed in the epidural or subdural spaces, the present disclosure describes the possibility of intradural electrodes.

In comparison to conventional techniques the systems and methods described herein may provide the ability to electrophysiologically “map” the temporal lobe from anterior and posterior sides, as the described techniques may be used access the inferior petrosal sinus and cavernous sinus (venous).

In some embodiments, the endovascular EEG device may be shaped as a microwire, stent, or other configuration implanted in the inferior petrosal and cavernous venous sinuses. Access to the inferior petrosal and/or cavernous venous sinuses may be achieved via the axillary, basilic or cephalic vein, or subclavian. In some embodiments, transcutaneous connectors may connect with leads connected to the electrode array positioned in the dura. In some embodiments, the transcutaneous connectors may connect with an external wearable device, computing system and the like. In some embodiments, the resulting implanted intradural device may be configured to be within the brain for up to 30 days.

In some embodiments, an endovascular neural electrode array may be implanted within branches of the external carotid artery. In some embodiments, the endovascular neural electrode array can be positioned within the middle meningeal artery by trans-arterial access from the radial or femoral routes to the middle meningeal artery.

Alternatively, the middle meningeal artery may be accessed percutaneously via the superficial temporal artery. Vascular navigation of the endovascular neural electrode array may be restricted to the external carotid system, which may result in an enhanced safety profile. In some embodiments, vascular access may be achieved via a stent deployment mechanism including a sheath, catheter, wire, and stent. In some embodiments the electrode array will be configured on a substrate constituting a wire, catheter, or stent.

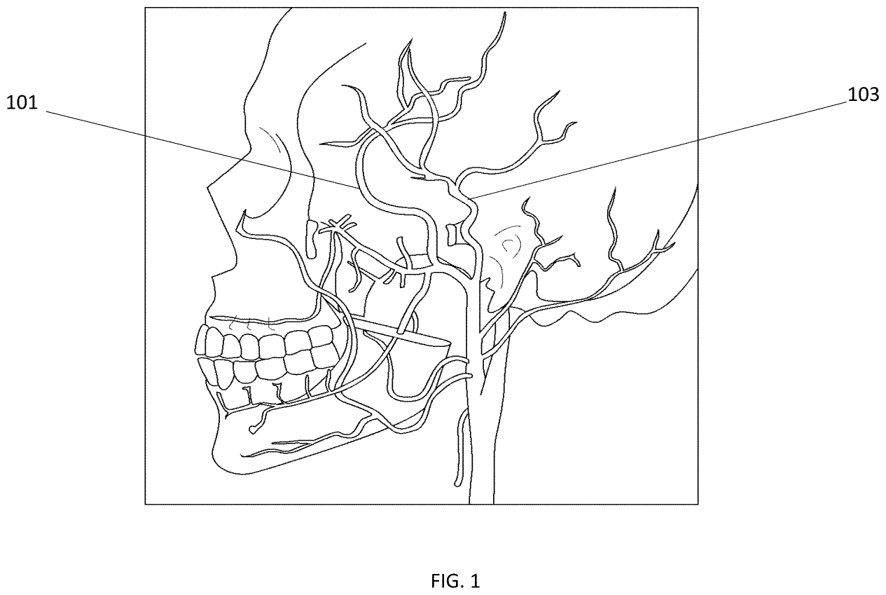

As illustrated in FIG. 1, the superficial temporal artery 103 is a branch of the external carotid artery, located in the scalp under the skin and over/outside the skull. The middle meningeal artery 105 is another branch of the external carotid artery and is located within the skull. A catheter or wire can be advanced via the superficial temporal artery 103 into the middle meningeal artery as both arteries are branches of the external carotid artery. A recording device within the middle meningeal artery 105 will be located on the surface of the brain, with significant advantages in recording compared to the traditional EEG recording from the scalp and with comparable signal characteristics with respect to conventional intradural electrocorticography, ECoG. An endovascular neural electrode device placed with the middle meningeal artery 105 has the unique advantage of being located within the dura, referred to herein as “intradural.” Unlike conventional methods of placing electrodes on the surface of the brain, which require removing parts of the skull, no bone removal is required to place these intradural electrodes.

In some embodiments, the endovascular neural electrode array can be placed within the middle meningeal artery 105 for short term recording (hours) via the arteries of the upper or lower extremity using standard interventional techniques. Using this approach, the device may be advanced in the middle meningeal artery 105 via the common and external carotid artery.

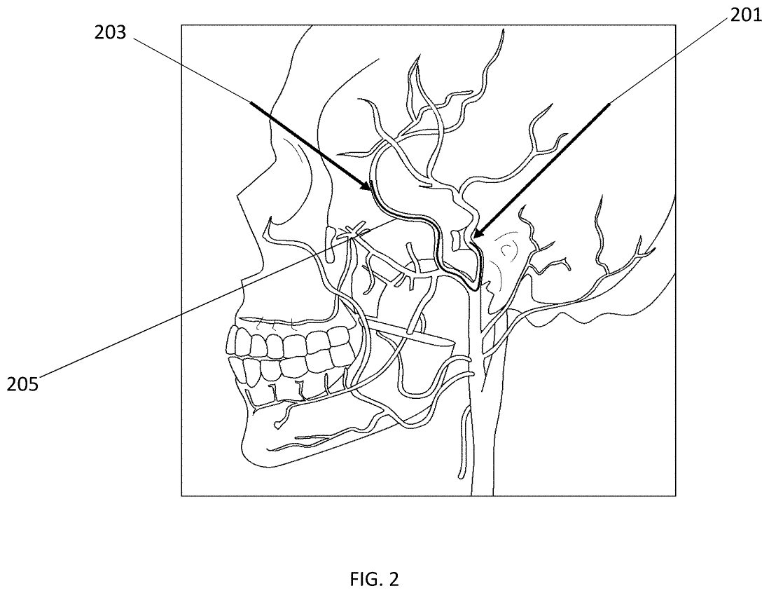

FIG. 2 illustrates an approach to the placement of “intradural” device. After a small incision is made in the scalp the superficial temporal artery 201 may be catheterized. An electrode array (wire or catheter) may then be navigated into the middle meningeal artery 203 using fluoroscopic guidance via path 205. As the electrode array is navigated into the middle meningeal artery 203 the electrode array will pass through the foramen spinosum (FS), a small, naturally occurring hole in the base of the skull; this hole permits passage of a device through the skull to the surface of the brain without removing any bone. In some embodiments, the location and positioning of the device within the middle meningeal artery may be confirmed with injection of iodinated contrast.

In some embodiments, the approach illustrated in FIG. 2 may allow for minimally invasive access to the intradural location and for long term recording (days to weeks). As the major arteries are not involved the procedure is safe for long term recording. With this approach there is no risk of stroke due to the long-term recording.

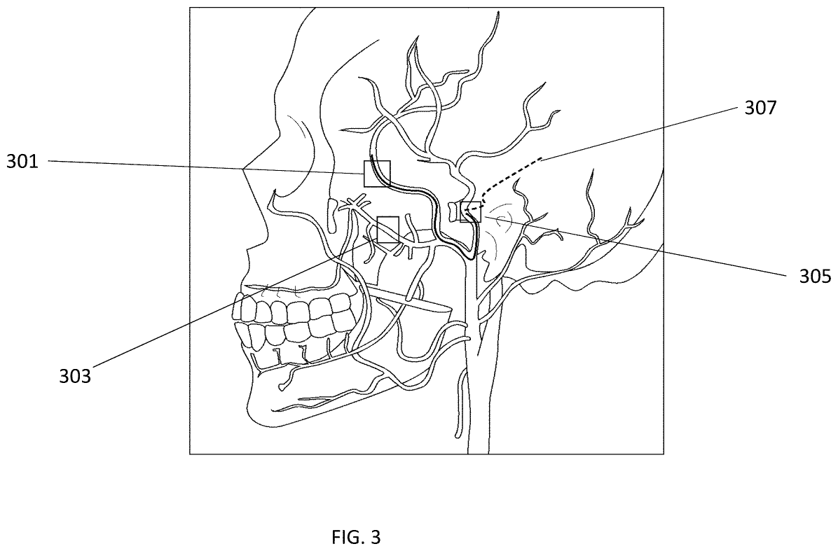

FIG. 3 illustrates the placement of a intradural device. As illustrated, endovascular elements scaffolds, electrodes, and connectors from electrodes to scaffolds may be positioned at location 301. A connector 303 may connect the scaffold to the transcutaneous site. The arteriotomy/percutaneous cannulation site, and transcutaneous access port are positioned at location 305. The device may connect to a wearable computer system 307, positioned outside of the body, along the scalp or behind the ear.

In contrast to conventional systems where there may have been catheterization of external carotid artery branches in the neck via cannulation of the superficial temporal artery, the present disclosure utilizes the cannulation of the superficial temporal artery and the subsequent navigation into the middle meningeal artery for placement of a recording device (wire or catheter).

While this approach allows placement of a device on the surface of the brain, this can be achieved via a minimally invasive approach, without the need of open surgery.

In some embodiments the endovascular neural electrode array may include a microwire electrode that is configured for placement in the external carotid artery branches. In some embodiments, the microwire electrode may include a diameter between about 200-400 micrometers. In some embodiments, a microwire electrode may have an optimal diameter of 0.36 mm. The microwire electrodes may have a recording length of 20-40 mm. Accordingly, from their position in the dura, the microwire electrodes may be able to record from the frontal, temporal, and parietal lobes.

Microwire electrodes may be composed of various materials, including, but not limited to gold, silver, platinum, and platinum-iridium.

The endovascular neural electrode array may connect to a transcutaneous or tunneled subcutaneous connectors having integrated systems for power, control, data, and communication and the like. In some embodiments, the external systems may be positioned along the scalp or behind the ear.

In the present disclosure, dedicated vascular closure devices for the superficial temporal artery with electrodes in place and after electrode removal are not needed, and hemostasis can be achieved using gentle manual compression for several minutes.

In some embodiments, the endovascular neural electrode array may be implanted by first creating a port for transcutaneous connection and soft tissue anchors. For example, access may be gained by the superficial temporal artery. During placement and manipulation of the electrodes a short sheath (e.g., 2-3 French and 3-5 cm in length) can be used for initial cannulation and procedural access.

Post-procedurally, the access sheath can be exchanged for a transcutaneous port containing a hemostatic plug used to maintain hemostasis and mechanical stability of electrode leads tunneled out of the superficial temporal artery, through the skin, for connection to the external computer, power source, and recording system.

In some embodiments, the hemostatic plug may be composed of collagen. The hemostatic plug may be positioned within the artery to ensure hemostasis around the existing connector. A sterile adhesive patch may be applied to the arteriotomy in order to maintain sterility and relieve strain from the existing connector.

In some embodiments, access to the external carotid branches, including the middle meningeal artery, may be obtained via direct puncture of the superficial temporal artery.

In some embodiments, the microwire and catheter length may be tapered and adapted to the distance between the superficial temporal access point to the target in the distal middle meningeal artery.

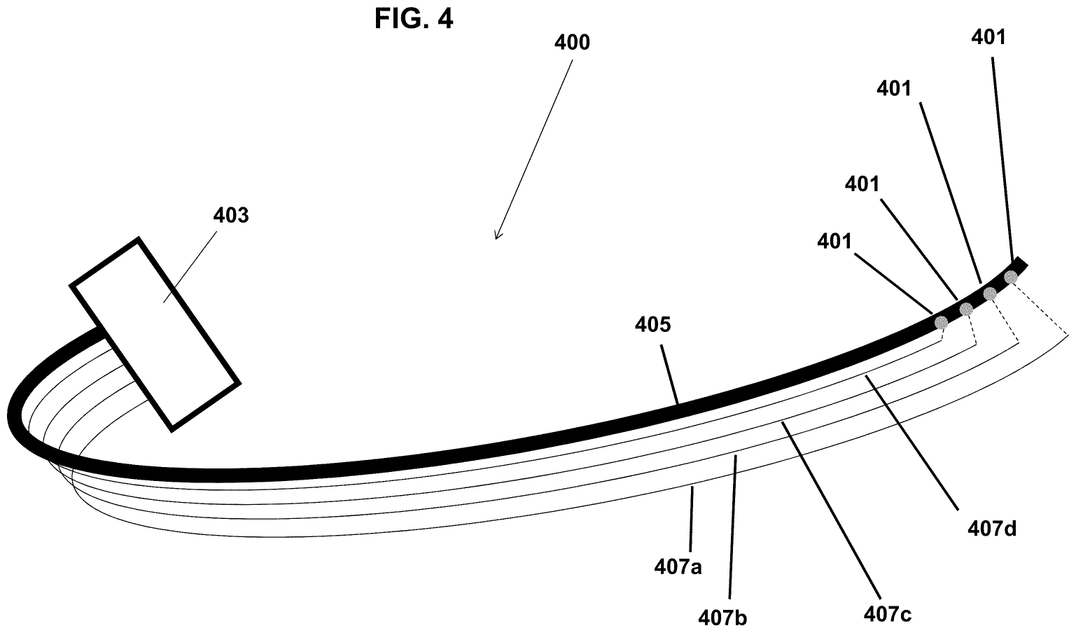

In some embodiments, microwire-based electrode arrays, such as that illustrated in FIG. 4, may be placed within the middle meningeal artery. These arrays will be constructed as composite wires (wire bundles 405), and the pliability of these wires 407a, 407b, 407c, 407d may vary along their length to achieve optimal vascular access through the arterial tree leading to the middle meningeal artery and its branches. The electrode array 400 can include multiple electrodes 401 positioned the distal (implanted) end of the electrode array. Each electrode 401 may be connected to an amplifier and recording apparatus 403 located at a distance spaced apart from the electrodes 401. The electrodes 401 may be connected to a multiplexing element located within the body, or they may be individually routed to amplifiers located outside the body. Traces 407a, 407b, 407c, 407d to each electrode 401 are separately insulated. In some embodiments, the traces 407a, 407b, 407c, 407d may be bundled together as a single composite “wire” 405 which is coated with insulated and hydrophilic coatings. In some embodiments the composite “wire” 405 may be 6-35 thousandths of an inch in diameter and have a length of approximately 10-30 cm. The diameter of the composite wire 405 may taper toward the distal tip. In some embodiments such a composite “electrode wire” could have many more than four electrodes 401. There may be from 8-256 electrodes, or possibly hundreds, thousands, or more electrodes 401 on the array. The electrode contacts are typically 5-20 microns but possibly up to 200-500 microns in diameter, and are made of conductive materials such as gold, silver, platinum, or platinum-iridium. Typical impedances are less than 25 Ohm but may be from 25 Ohm to 25 kOhm. Electrode surfaces may be coated for optimization of surface properties by materials such as PEDOT. Interelectrode spacing is similar to the electrode diameter, on the order of 5-500 microns.

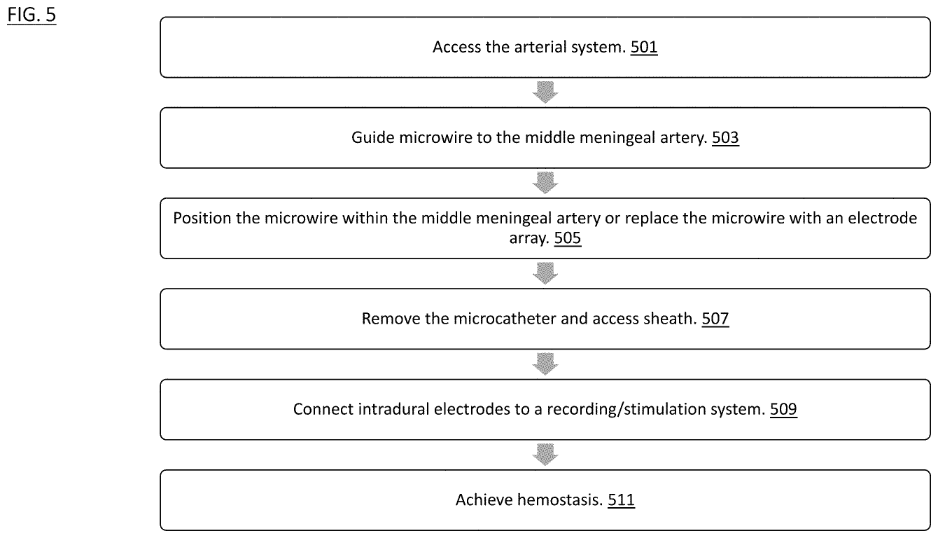

FIG. 5 illustrates a method for deploying intradural electrodes within the middle meningeal artery. In a first step 501, arterial access is achieved. This may be through traditional transradial or transfemoral access to the carotid artery and then to the external carotid artery and middle meningeal artery via the internal maxillary branch. Alternatively, direct percutaneous cannulation of the superficial temporal artery in the scalp may be achieved possibly using ultrasound or other guidance methods. In a second step 503, the microwire may be guided to the middle meningeal artery. This may utilize techniques including, but not limited to, fluoroscopy and contrast dye injection as needed, while the microwire and microcatheter are navigated into the middle meningeal artery. In a third step 505, electrodes may be placed in the middle meningeal artery. For example, the microwire may be positioned in the middle meningeal artery. Alternatively, the microwire may be exchanged for the electrode array. Array placement may be confirmed by fluoroscopy and/or initial electrophysiologic recordings. The electrode array can be moved dynamically, in real time, based on the visualized location as well as the electrophysiologic signals. The patient may be awake, sedated, or under general anesthesia, as the clinical situation demands. In a fourth step 507, the procedure may be closed. In the fourth step, the microcatheter and access sheath may be removed, leaving the electrode array in place, with the lead wire exiting the skin at the vascular access point if long-term recording is desired. In a fifth step 509, the intradural electrodes may be connected to a recording system. For example, if long-term recording after the procedure is desired, the distal ends of the electrode leads may be connected to a computational unit. This unit and the distal ends of the leads may be secured to the body externally, for example by placement behind the ear, or it may be surgically placed under the skin so that no components remain exposed. In the former case sterile adhesive is used to secure the computing unit and the distal end of the electrode array. In a sixth step 511, hemostasis may be achieved, for example, via manual compression for several minutes.

In some embodiments, the electrode array may be between about 0.2 to 0.4 mm in diameter, and have a recording length of 20-40 mm. The electrode array may access the dura via either the axillary, basilica, cephalic, or subclavian veins.

Embodiments of the device built in accordance with the present disclosure may be used to map, record, and/or stimulate the brain and nervous system to diagnose and treat a variety of conditions. Examples of conditions may include epilepsy/seizure disorders, paralysis associated with stroke or spinal cord injury, movement disorders such as Parkinson's disease and essential tremor, chronic pain disorders, neuroendocrine and hypothalamic disorders (including obesity), neuropsychiatric disorders and addiction, and human-to-computer interfaces, and the like.

### Applications to Epilepsy

The disclosed systems and methods may be used for the detection and/or treatment of epilepsy, among other conditions. Fifty million people in the world have epilepsy, and there are between 16 and 51 cases of new-onset epilepsy per 100,000 people every year. A community-based study in southern France estimated that up to 22.5% have drug resistant epilepsy. Patients with drug-resistant epilepsy have increased risks of premature death, injuries, psychosocial dysfunction, and reduced quality of life.

Approximately three million American adults reported active epilepsy in 2015. Active epilepsy, especially when seizures are uncontrolled, poses substantial burdens because of somatic, neurologic, and mental health comorbidity; cognitive and physical dysfunction; side effects of anti-seizure medications; higher injury and mortality rates; poorer quality of life; and increased financial cost. The number of adults reporting that they have active epilepsy has significantly increased from 2010 (2.3 million) to 2015 (3 million), with about 724,000 more cases identified from 2013 to 2015. 20-30% of patients with epilepsy have a medically and socially disabling seizure disorder which leads to increased morbidity and mortality, depression and physical trauma.

“Medically intractable” patients by definition have failed at least two antiepileptic medications. The chance of becoming “seizure-free” after failing two appropriate seizure medications is extremely low. Severe medication side effects may also be an indication for surgery. To determine if a patient is a candidate for epilepsy surgery, an extensive evaluation is undertaken, including video EEG telemetry, anatomical (MRI) and functional (positron emission tomography (PET) or single photon emission computerized tomography (SPECT) imaging) and neuropsychological testing, as well as angiographically assisted pharmacologic testing (“Wada” testing or intracarotid sodium amobarbital procedure), electrocorticography (ECoG), and stereo-electroencephalography (sEEG).

Conventional EEG is an important diagnostic test in the evaluation of a patient with epilepsy. During a conventional EEG test, electrical activity is recorded from standard sites on the scalp according to the standard 10-20 system of electrode placement. The EEG signal depends upon differential amplification between voltage recordings from paired sites on the scalp, and it is in turn recorded as a voltage tracing, with each electrode pair defining an output “channel.” Despite the widespread availability and ease of usage of EEG testing there two major limitations: (1) intermittent EEG changes (such as seizures) can be infrequent and may not appear during the period of recording, which may range from 30 minutes to 3 days, and (2) some highly epileptogenic areas, such as the medial temporal lobes, are not well explored by the scalp electrodes and so the diagnostic yield is suboptimal.

In an alternative to conventional EEG, other electrodes have been developed to engage with the sphenoidal, nasopharyngeal, ear canal, and/or mandibular notch, in order to aid with the diagnosis of seizures. However, these alternatives are often uncomfortable to the patient and prone to artifacts and misinterpretation, providing limited usage and yield in practical settings.

For patients requiring more invasive evaluation, conventional practice involves the use of intracranial EEG, or electrocorticography (ECoG). This approach requires surgical implantation of EEG electrodes in order to better lateralize and localize the seizure focus. Electrodes placed on the brain surface and directly in the brain can be used to map seizure activity. Placement of these electrodes requires craniotomy (or at least multiple burr holes through the skull) for surgical implantation, while the patient needs to remain hospitalized for 3-5 days or longer while the electrodes are recording. Then, a second procedure is necessary to remove the electrodes and restore the craniotomy defect (yet a third surgery would be required to remove or ablate the seizure focus). However, it is possible that even after the surgical implantation, the location of a single seizure focus is not determined. The invasive nature of this procedure and the possible failure to identify and localize seizure origin indicates a need for more accurate and less invasive means of identifying and localizing seizure foci.

Embodiments of the present disclosure may include electrode arrays for neural recording and stimulation that can be deployed using minimally invasive endovascular surgical techniques, accessing the brain through arteries and veins into the brain.

In some embodiments, the electrode array may be implanted within the dura for minutes to weeks long durations for diagnostic procedures, inpatient monitoring, and/or outpatient monitoring post-procedure. The electrode array may be catheter-based, wire-based, and/or stent-based.

In some embodiments, the disclosed electrode arrays may be configured for deployment in the dura matter of the brain and configured to record and/or stimulate from adjacent brain tissue. In some embodiments, the electrode arrays may be designed for deployment within the blood vessels of the dura mater of the brain, including the middle meningeal artery and its branches. The electrode arrays may be connected to intravascular leads and connectors which are routed through the vascular system, for transvascular and either subcutaneous connection to an implanted computer system, or external (transcutaneous) connection to an externally wearable computer system responsible for control of power, system control, data storage, and data transmission (wired or otherwise).

The system also comprises a mechanism for stable percutaneous cannulation of the superficial temporal artery, and deployment of intradural electrodes via branches of the external carotid artery, without entering the arteries that primarily supply the brain itself.

In some embodiments, recording from the intradural electrode array may provide access to regions of the brain previously not possible to record from. Further, in contrast to conventional systems the intradural recordings may provide higher quality recordings than extradural recordings. At the same time, because the arteries accessed for intradural recordings are very safe to enter as they do not supply the brain itself (only the membranes covering the brain), and their occlusion does not result in neurologic impairment, intradural recordings may be safer than the subdural recordings often used in conventional electrocorticography (ECoG).

For example, when compared to conventional subdural arrays, the intradural arrays disclosed herein may be less invasive, with an improved safety profile that is better because they do not require opening the dura for placement. Opening the dura for placement is associated with exposure of brain and increases the risk of infection and other complications for the patient.

In some embodiments, the disclosed systems may be positioned within the dura of the brain and the electrodes may be positioned along small intradural arteries. Accordingly, the disclosed systems may record and function similarly to electrocorticography electrodes and may be positioned very close to the cortical surface. However, unlike ECoG, the disclosed systems would not require opening of the dura and exposing the brain, which comes with associated risks.

Additionally, in some embodiments, the disclosed systems include a mechanism for the stable percutaneous cannulation of the superficial temporal artery, and deployment of intradural electrodes via branches of the external carotid artery, without entering the arteries that primarily supply the brain itself. In comparison to conventional systems this may provide 1 improvements in the safety profile.

In another embodiment, the disclosed systems and methods may be considered as alternatives to scalp EEG electrodes but provide better recording quality due to being in closer contact with the brain (similar to conventional ECoG electrodes, which, unlike scalp EEG electrodes, do not record through intervening scalp and skull).

In some embodiments, placement of the device within the middle meningeal artery and the dura (“intradural”) can be maintained long term, as the risk of complications due to occlusion of this vessel is negligible. If fact, the middle meningeal artery is often sacrificed during surgical procedures (craniotomy) or permanently occluded through endovascular embolization in patients with history of intracranial hemorrhage or brain tumor.

In some embodiments, placement of the intradural device is via a small incision in the scalp. This approach will allow for safe delivery of the device in the middle meningeal artery via the superficial temporal artery. The device can be left in the middle meningeal artery for up to 30 days or possibly longer without associated risk of stroke (as opposed to the other intra-arterial locations).

While the disclosure has been described in connection with certain embodiments, it is to be understood that the disclosure is not to be limited to the disclosed embodiments but, on the contrary, is intended to cover various modifications and equivalent arrangements included within the spirit and scope of the disclosure, which scope is to be accorded the broadest interpretation so as to encompass all such modifications and equivalent structures as is permitted under the law.

1. An implantable medical device comprising:
a linear array of electrodes configured for insertion between layers of a dura membrane of a brain, the linear array configured to record or stimulate electrical activity in brain tissue, and
an insulated electrode trace connecting each of electrodes in the linear array.

2. The implantable medical device of claim 1, wherein each of the electrodes comprises at least one of gold, silver, platinum, or platinum-iridium and each of the electrodes has a diameter between about 5 to 25 microns, or 25 to 250 microns.

3. The implantable medical device of claim 1, wherein the insulated electrode trace comprises a diameter between about 6 to 35 thousandths of an inch in diameter and has a length between about 10 to 30 cm.

4. The implantable medical device of claim 1, wherein the diameter of at least one of the insulated electrode trace or plurality of wire traces contained within the insulated electrode trace tapers proximate the electrode.

5. The implantable medical device of claim 1, wherein each electrode is connected to a multiplexing element.

6. The implantable medical device of claim 1, wherein each electrode is connected to an amplifier located outside of the body.

7. The implantable medical device of claim 1, wherein the linear array of electrodes is positioned at the middle meningeal artery.

8. The implantable medical device of claim 1, wherein the linear array of electrodes is positioned proximate the dural venous sinuses.

9. The implantable medical device of claim 1, wherein a surface of each of the electrodes is coated by poly(3,4-ethylenedioxythiophene) polystyrene sulfonate (PEDOT).

10. The implantable medical device of claim 1, wherein the inter-electrode spacing distance between adjacent electrodes in the linear array is between about 5-500 microns.

11. The implantable medical device of claim 1, wherein the insulated electrode trace connects to a long-term recording system or a stimulation system.

12. The implantable medical device of claim 1, wherein the an insulated electrode trace comprises a hydrophilic coating.

13. A method for operating intradural electrodes comprising:
advancing a microwire through the external carotid artery to the middle meningeal artery;
positioning electrodes between layers of the dura membrane via the middle meningeal artery; and
recording or stimulating the brain tissue, wherein the microwire includes a linear array of electrodes configured for insertion between layers of the dura membrane of the brain, the linear array comprising a plurality of electrodes configured to record or stimulate electrical activity in brain tissue,
wherein each of the electrodes is connected to the linear array via an insulated electrode trace.

14. The method of claim 13, wherein positioning electrodes within the layers of the dura further comprises applying fluroscopy or contrast dye techniques.

15. The method of claim 13, further comprising moving the position of electrodes between layers of the dura membrane.

16. The method of claim 13, wherein the plurality of insulated electrode traces form a bundle coated with hydrophilic coatings.

17. A method for deploying intradural electrodes comprising:
advancing a microwire through the external carotid artery to the middle meningeal artery;
advancing an electrode array over the microwire to the middle meningeal artery;
removing the microwire; and
recording or stimulating the brain tissue about the electrode array; wherein the electrode array comprises a plurality of electrodes configured to record or stimulate electrical activity in brain tissue and the plurality of electrodes are on an insulated substrate.

18. The method of claim 17, further comprising positioning the electrode array within the middle meningeal artery using at least one of fluroscopy, and contrast dye techniques.

19. The method of claim 17, further comprising moving the position of the electrode array between layers of the dura membrane.

20. The method of claim 17, wherein electrode array is implanted within the brain for weeks.

21. The method of claim 17, further comprising:
advancing a microcatheter to recover and remove the electrode array.
